(19) United States
(12) Patent Application Publication
(10) Pub. No.: US 2011/0229531 A1
(43) Pub. Date: Sep. 22, 2011

(54) ANTI-PROTOZOA COMPOUNDS

(76) Inventors: Fiona Henriquez, Paisley (GB); Craig Roberts, Glasglow (GB)

(21) Appl. No.: 13/063,858

(22) PCT Filed: Sep. 15, 2009
(86) PCT No.: PCT/GB2009/051197

§ 371 Date: Jun. 3, 2011

(51) Int. Cl. A61K 9/00; C07H 21/04; C07D 249/14; A61K 31/711; A61K 31/4196; A61K 31/505; A61K 31/53; A61K 31/7016; C12Q 1/02; G01N 33/573; A61K 31/7115; C07H 21/02; A61P 33/02; A61P 33/04; A45C 11/04

(52) U.S. Cl. 424/400; 536/23.2; 548/264.8; 514/44 A; 514/383; 514/275; 514/245; 514/53; 435/29; 435/7.4; 206/5.1

(57) ABSTRACT

Use of a compound in the control of growth of at least one organism of the order of Amoebida, wherein said compound is capable of modulating the histidine biosynthesis pathway, and/or the methionine biosynthesis pathway and/or a pathway branching from the histidine biosynthesis pathway and/or methionine biosynthesis pathway in said organism.

### FIELD OF THE INVENTION

The present invention relates to compounds that modulate the histidine biosynthesis pathway, and/or the methionine biosynthesis pathway and/or a pathway branching from the histidine biosynthesis pathway and/or a pathway branching from the methionine biosynthesis pathway in the Amoebida order, the use of such compounds in the treatment or prevention of diseases caused or contributed to by members of the Amoebida order and non-therapeutic uses.

### BACKGROUND OF THE INVENTION

Acanthamoeba species are free-living protozoa found throughout the environment. Acanthamoeba species have a biphasic life cycle and exist as either active trophozoites or dormant cysts. They are found ubiquitously in the environment including tap-water, water tanks, contact lens cases and eyewash solutions.

Acanthamoeba can parasitise humans causing severe debilitating eye disease in healthy individuals called Acanthamoeba keratitis and the death threatening disease granulomatous Acanthamoeba encephalitis (GAE) in the immunocompromised.

Incidence of Acanthamoeba keratitis has been estimated to be 1 per 30000 contact lens wearers per year, although a separate study found that it could be as high as 20, 3.3 and 1.1 per 10,000 for extended wear, daily disposable and rigid gas permeable lenses, respectively.

“One step” contact lens solutions are not capable of eliminating Acanthamoeba and only the correct use of “two step” solutions containing hydrogen peroxide provide a small protection against Acanthamoeba transmission to the eye. If an individual becomes infected with Acanthamoeba, treatment is often very painful and extremely arduous, involving hospitalisation and often resulting in a necessary corneal transplant. Reactivation can also occur in the transplanted cornea thus resulting in the loss of sight.

New compounds which are effective against the Amoebida order are urgently required.

### SUMMARY OF THE INVENTION

Whilst the histidine pathway has previously been shown to be present in bacteria, fungi and plants, it was not considered to be present in protozoa such as Acanthamoeba. The inventors have surprisingly identified the histidine biosynthesis pathway in Acanthamoeba and demonstrated both that Acanthamoeba growth can be inhibited by known inhibitors of the histidine pathway in a dose dependent manner and that inhibition can be ablated in a dose dependent manner by the addition of histidine to the Acanthamoeba growth medium.

Accordingly, in a first aspect of the present invention there is provided a use of a compound for modulating the histidine biosynthesis pathway and/or any pathway branching from the histidine biosynthesis pathway in the Amoebida.

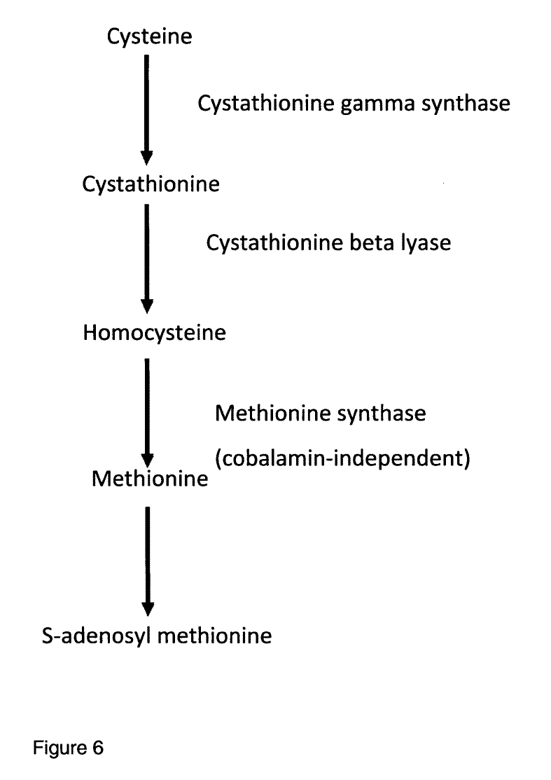

Bacteria, fungi and plants can synthesise methionine from cysteine in a cobalamin-independent manner (FIG. 6). The inventors have established that Acanthamoeba castellanii also possesses this metabolic pathway, as they have identified that A. castellanii expresses two genes involved in methionine biosynthesis, methionine synthase and cystathione gamma synthase and the inventors have established that A. castellanii trophozoites can be maintained in methionine-free defined medium and thus the cobalamin-independent methionine biosynthesis pathway can be exploited for anti-microbial therapy. Thus, Acanthamoeba growth can also be inhibited by inhibitors of the methionine pathway in a dose dependent manner and that inhibition can be ablated in a dose dependent manner by the addition of methionine to the Acanthamoeba growth medium.

Accordingly, in a second aspect of the present invention there is provided a use of a compound for modulating the methionine biosynthesis pathway and/or any pathway branching from the methionine biosynthesis pathway in the Amoebida.

In a third aspect of the invention there is provided a kit comprising
a compound for modulating, for example inhibiting, the histidine biosynthesis pathway and/or a pathway branching from the histidine biosynthesis pathway, and
a compound for modulating, for example inhibiting, the methionine biosynthesis pathway and/or a pathway branching from the methionine biosynthesis pathway
in Amoebida, in particular in Acanthamoeba castellanii and Acanthamoeba polyphaga, for separate, simultaneous, or sequential administration of i) and ii). The kit may be used to sanatize products such as eye prosthesis, a contact lens, storage holders for eye prosthesis or contact lens, in artificial tear formulations or other eye care products, and in cleaning formulations. Alternatively, the kit can be used in the treatment of diseases caused by Amoebida, in particular Acanthamoeba castellanii and Acanthamoeba polyphaga.

The Amoebida is an order within the Phylum Protozoa, which comprises the Vahlkampfidae, the Pelomyxidae, the Paramoebidae, the Mastigamoebidae, the Hartmannellidae, the Flabellulidae, the Entamoebidae, the Amoebidae and the Acanthamoebidae families.

Histidine and methionine are essential amino acids in humans and other mammals. This means that humans do not possess such biochemical pathways and that histidine and methionine must be acquired from the diet. In view of this, the histidine biosynthesis pathway and the methionine biosynthesis pathway present advantageous drug targets, as an anti-Acanthamoeba compound designed to inhibit growth by targeting such a pathway would be expected to have little or no side effect on the human host.

In embodiments of the invention, there is provided the use of a compound for modulating the histidine biosynthesis pathway, and/or the methionine biosynthesis pathway and/or a pathway branching from the histidine biosynthesis pathway and/or a pathway branching from the methionine biosynthesis pathway in the Acanthamoebidae. In particular embodiments, there is provided the use of a compound to modulate said pathways in Acanthamoeba, specifically for example Acanthamoeba castellanii and Acanthamoeba polyphaga.

The term “branching” may be taken to mean a pathway that metabolises a particular product derived from the histidine or methionine biosynthesis pathway.

Use of a compound capable of modulating the histidine biosynthesis pathway, and/or the methionine biosynthesis pathway and/or a pathway branching from the histidine biosynthesis pathway and/or a pathway branching from the methionine biosynthesis pathway may be expected to modulate the production of histidine/methionine respectively by the member of the Amoebida order and will likely have an effect upon the ability of the Amoebida to grow and/or survive.

Use of compounds that can modulate the histidine biosynthesis pathway, and/or the methionine biosynthesis pathway and/or a pathway branching from the histidine biosynthesis pathway and/or a pathway branching from the methionine biosynthesis pathway may modulate said pathway to an extent that the growth of the Amoebida is inhibited (i.e. microbistatic (amoebidastaic)), and/or alternatively may cause the Amoebida to die (i.e. microbicidal (amoebidacidal).

In embodiments a compound used in the invention may be an inhibitor of an enzyme or enzymes of the histidine biosynthesis pathway, and/or the methionine biosynthesis pathway and/or a pathway branching from the histidine biosynthesis pathway and/or a pathway branching from the methionine biosynthesis pathway.

In embodiments, the compound for use in the invention may modulate the histidine biosynthesis pathway, and/or the methionine biosynthesis pathway and/or a pathway branching from the histidine biosynthesis pathway and/or a pathway branching from the methionine biosynthesis pathway, by modulating the activity and/or expression of an enzyme of the histidine biosynthesis pathway, and/or the methionine biosynthesis pathway and/or a pathway branching from the histidine biosynthesis pathway and/or a pathway branching from the methionine biosynthesis pathway.

Suitably, in embodiments, a compound for use in the invention may be an enzyme modulator, for example an enzyme inhibitor or a modulator of enzyme expression.

In embodiments, a modulator of enzyme expression may include an oligonucleotide sequence complementary to a section of the genome of a member of the Amoebida order, which section encodes enzymes involved in the histidine biosynthesis pathway, and/or the methionine biosynthesis pathway and/or a pathway branching from the histidine biosynthesis pathway and/or a pathway branching from the methionine biosynthesis pathway. Suitably, in embodiments, the oligonucleotide may be RNAi or antisense. The antisense molecules may be natural or synthetic. Synthetic antisense molecules may have chemical modifications from native nucleic acids. Antisense molecules inhibit gene expression through various mechanisms, e.g. by reducing the amount of mRNA available for translation, through activation of RNAse H, or steric hinderance. One or a combination of antisense molecules may be administered, where a combination may comprise multiple different sequences. Antisense oligonucleotides will generally be about 7, usually at least about 12, more usually at least about 16 nucleotides in length, and usually not more than about 50, preferably not more than about 35 nucleotides in length.

Preferred RNAi agents of and for use in the invention are between 15 and 25 nucleotides in length, preferably between 19 and 22 nucleotide, most preferably 21 nucleotides in length. In embodiments, the invention provides methods of employing an RNAi agent to modulation the expression, preferably reducing expression of an enzyme in the histidine and/or methionine biosynthetic pathway. By reducing expression is meant that the level of expression of a target gene or coding sequence is reduced or inhibited by at least about 2-fold, usually by at least about 5-fold, e.g. 10-fold, 15-fold, 20-fold, 50-fold, 100-fold or more, as compared to a control. The RNAi agents that may be employed in preferred embodiments of the invention are small ribonucleic acid molecules (also referred to herein as interfering ribonucleic acids), that are present in duplex structures, e.g. two distinct oligoribonucleotides hybridized to each other or a single ribooligonucleotide that assumes a small hairpin formation to produce a duplex structure. Preferred oligoribonucleotides are ribonucleic acids of not greater than 100 nt in length, typically not greater than 75 nt in length. Where the RNA is siRNA, the length of the duplex structure typically ranges from about 15 to 30 bp, usually from about 20 to 29 bp, most preferably 21 bp. Where the RNA agent is a duplex structure of a single ribonucleic acid that is present in a hairpin formation, i.e., a shRNA, the length of the hybridized portion of the hairpin is typically the same as that provided about for the siRNA of agent or longer by 4-8 nucleotides.

In certain embodiments, instead of the RNAi agent being an interfering ribonucleic acid, e.g. an siRNA or shRNA as described above, the RNAi agent may encode an interfering ribonucleic acid. In these embodiments, the RNAi agent is typically a DNA that encodes the interfering ribonucleic acid. The DNA may be present in a vector.

The RNAi can be administered to the host using any suitable protocol known in the art. For example, the nucleic acids may be introduced into tissues or host cells by viral infection, microinjection, fusion of vesicles, particle bombardment, or hydrodynamic nucleic acid administration.

DNA directed RNA interference (ddRNAi) is an RNAi technique which may be used in the methods of the invention.

In other embodiments, morpholinos may be used to down-regulate gene expression using anti-sense technology. In such embodiments, phosphordiamidate morpholino oligo (PMO) can be used to knock down gene expression in at least one, preferably both of the histidine and methionine biosynthesis pathways and/or a pathway branching therefrom. This can typically be achieved by the PMO sterically blocking the RNA. The phosphordiamidate morpholine oligo may be peptide conjugated. Such can potentially be used as a mode of therapy for Acanthamoeba keratitis, having already been successfully demonstrated to inhibit HSV-1 infections in the eye (Moerdyk-Schauwecker et al, Antiviral Res. 2009).

In embodiments, the oligonucleotide sequences may be DNA or RNA which interfere with the functional sequences encoding the enzymes of the histidine biosynthesis pathway, and/or the methionine biosynthesis pathway and/or a pathway branching from the histidine biosynthesis pathway and/or a pathway branching from the methionine biosynthesis pathway in the Amoebida, and/or oligonucleotide sequences which interfere with the RNA transcripts of the functional enzyme genes, for example interfering RNA (iRNA), short interfering RNA (siRNA) or antisense oligonucleotides.

Algorithms such as BIOPRED57 may be used to computationally predict siRNA sequences that have an optimal knockdown effect for a given gene of the histidine biosynthetic pathway and/or the methionine biosynthesis pathway.

Enzyme inhibitors of the histidine biosynthesis pathway or a pathway branching from the histidine biosynthesis pathway are well known to one of ordinary skill in the art, for example 3-amino-1,2,4-triazole (3AT). Enzyme inhibitors may be classified as, for example, specific, non-specific, reversible, non-reversible, competitive and non-competitive.

Compounds potentially useful in the modulation of the methionine biosynthesis pathway or a pathway branching from the methionine biosynthesis pathway may include inhibitors of cystathionine gamma synthase such as dl-E-2-amino-5-phosphono-3-pentenoic acid, 3-(phosphonomethyl)pyridine-2-carboxylic acid and 5-carboxymethylthio-3-(3′-chlorophenyl)-1,2,4-oxadiazol.

In the present invention any compound capable of inhibiting any enzyme or enzymes of the histidine biosynthesis pathway, and/or the methionine biosynthesis pathway and/or a pathway branching from the histidine biosynthesis pathway and/or a pathway branching from the methionine biosynthesis pathway, may potentially be used to modulate the production of histidine/methionine, respectively, in the Amoebida.

In particular embodiments, a compound for use in the invention may be a small organic molecule capable of modulating the function of an enzyme or enzymes of the histidine biosynthesis pathway, and/or the methionine biosynthesis pathway and/or a pathway branching from the histidine biosynthesis pathway and/or a pathway branching from the methionine biosynthesis pathway. Typically a small organic molecule may be an analogue of any of the substrates of the enzymes utilised in such pathways. Conveniently the substrate analogue may be modified in such a way so as to either inhibit and/or prevent the progression of a particular enzymatic reaction in the histidine biosynthesis pathway, and/or the methionine biosynthesis pathway and/or a pathway branching from the histidine biosynthesis pathway and/or a pathway branching from the methionine biosynthesis pathway.

In addition or alternatively, a compound for use in modulating the histidine biosynthesis pathway, and/or the methionine biosynthesis pathway and/or a pathway branching from the histidine biosynthesis pathway and/or a pathway branching from the methionine biosynthesis pathway in the Amoebida, for example a small organic enzyme substrate analogue, may interact with a particular enzyme or enzymes or a substrate of the pathway, and in turn result in the production of a product which is incapable of being utilised by another enzyme of the histidine biosynthesis pathway, and/or the methionine biosynthesis pathway and/or a pathway branching from the histidine biosynthesis pathway and/or a pathway branching from the methionine biosynthesis pathway. In this way further enzymatic reactions will not be possible and the pathway will be unable to progress.

Suitable compounds for use in the present invention to modulate the histidine biosynthetic pathway and/or a pathway branching from the histidine biosynthetic pathway include 3-amino triazole (3-AT). A person of skill in the art will be familiar with other suitable compounds as previously determined, for example in relation to herbicides.

Advantageously the compounds for use in the present invention can also be used to prevent bacterial growth and thus may be used, for example as components in solutions, such as solutions for contact lenses, to prevent bacterial growth. They may also be used in antibiotic coatings on surgical instruments and in products such as medicated soaps. Accordingly, the present invention provides the non-therapeutic use of a compound of the invention in inhibiting bacterial growth. Also provided is a contact lens solution, eye care product or a medicated soap comprising a compound which can modulate the histidine biosynthesis pathway, and/or the methionine biosynthesis pathway and/or a pathway branching from the histidine biosynthesis pathway and/or a pathway branching from the methionine biosynthesis pathway in the Amoebida. Further, the present invention provides a surgical instrument or prosthesis having thereon an antibiotic coating comprising a compound which can modulate the histidine biosynthesis pathway, and/or the methionine biosynthesis pathway and/or a pathway branching from the histidine biosynthesis pathway and/or a pathway branching from the methionine biosynthesis pathway in the Amoebida, in particular in Acanthamoeba.

As will be appreciated, the determination of a further drug target in Amoebida, in particular in Acanthamoeba, provides for novel compositions comprising combinations of active agents which include a compound capable of modulating the activity and/or expression of an enzyme of the histidine biosynthesis pathway, and/or the methionine biosynthesis pathway and/or a pathway branching from the histidine biosynthesis pathway and/or a pathway branching from the methionine biosynthesis pathway and at least a second compound, wherein such novel compositions provide for an additive or synergistic anti-bacterial and/or anti-protozoa effect.

In preferred embodiments of the invention, the inhibitor of the histidine biosynthesis pathway and the inhibitor of the methionine biosynthesis pathway or another known inhibitor of the Amoebida or another pathogen can be administered in a potentiating ratio. The term “potentiating ratio” in the context of the present invention is used to indicate that the histidine inhibitor and the methionine inhibitor or the other inhibitor are present in such a ratio that the reduction in the number of Amoebida of the combination is greater than that of either inhibitor alone or of the additive activity that would be predicted for the combinations based on the activities of the individual inhibitors. Thus, in a potentiating ratio, the individual inhibitors act synergistically.

Synergism can be defined using a number of methods. For example, synergism may be defined as an RI of greater than unity. The RI may be calculated as the ratio of expected cell survival (Sexp, defined as the product of the survival observed with inhibitor A alone and the survival observed with inhibitor B alone) to the observed cell survival (Sobs) for the combination of A and B (RI=Sexp/Sobs). Synergism may then be defined as an RI of greater than unity. Other methods for determining synergy as would be known in the art may also be used. In preferred embodiments of the invention, the combined inhibitors are provided in concentrations sufficient to produce an RI of greater than 1.5, more preferably greater than 2.0, most preferably greater than 2.25.

Accordingly, a fourth aspect of the present invention provides a composition for use in modulating the histidine biosynthesis pathway, and/or the methionine biosynthesis pathway and/or a pathway branching from the histidine biosynthesis pathway and/or a pathway branching from the methionine biosynthesis pathway, wherein the composition comprises a compound capable of modulating the activity and/or expression of an enzyme of the histidine biosynthesis pathway, and/or the methionine biosynthesis pathway and/or a pathway branching from the histidine biosynthesis pathway and/or a pathway branching from the methionine biosynthesis pathway and at least a second compound.

In particular embodiments a second compound can modulate a further biosynthetic pathway present in Amoebida, in particular Acanthamoeba, specifically for example Acanthamoeba castellanii and Acanthamoeba polyphaga and/or act as an antimicrobial. In embodiments the second compound may not be active i.e. inhibit the growth or lead to the death of Amoebida, in particular Acanthamoeba, but may act against other pathogens such that the composition provides for an improved spectrum of action over the second compound alone.

By way of an example such a composition, may include a compound for use in modulating the histidine biosynthesis pathway, and/or the methionine biosynthesis pathway and/or a pathway branching from the histidine biosynthesis pathway and/or a pathway branching from the methionine biosynthesis pathway and a compound capable of modulating an enzyme of the shikimate pathway or a compound capable of modulating an enzyme of the folate pathway.

Compounds potentially useful in the modulation of the shikimate pathway would be well known to those of skill in the art and may include glyphosate, sulfosate and a modified precursor for EPSP Synthase.

Compounds potentially useful in the modulation of enzymes of the folate pathway would be well known to those of skill in the art and may include, for example, sulphonamides, pyrimethamine or any other compound known to interfere with folate synthesis including, for example, proguanil, cycloguanyl or trimethoprim and a number of derivatives, triazine exemplified by WR99210 and the prodrug PS-15 (Kinyanjui et al. 1999).

In embodiments of the invention, an antimicrobial compound for use in a combined composition with a compound capable of modulating the activity and/or expression of an enzyme of the histidine biosynthesis pathway, and/or the methionine biosynthesis pathway and/or a pathway branching from the histidine biosynthesis pathway and/or a pathway branching from the methionine biosynthesis pathway may include hexamidine, biguanide or chlorhexidine digluconate. Suitably, in such embodiments hexamidine may be provided at 0.1% and polyhexamethylene biguamide may be provided at 0.02%.

In addition to non-therapeutic uses, the present invention also provides therapeutic uses of compounds capable of modulating the activity and/or expression of an enzyme of the histidine biosynthesis pathway, and/or the methionine biosynthesis pathway and/or a pathway branching from the histidine biosynthesis pathway and/or a pathway branching from the methionine biosynthesis pathway.

Accordingly, a fifth aspect of the present invention provides the use of a compound capable of modulating the histidine biosynthesis pathway, and/or the methionine biosynthesis pathway and/or a pathway branching from the histidine biosynthesis pathway and/or a pathway branching from the methionine biosynthesis pathway in the Amoebida order, in particular Acanthamoeba, specifically for example Acanthamoeba castellanii and Acanthamoeba polyphaga for the preparation of a medicament for the treatment of diseases caused and/or contributed to by the Amoebida.

For example, diseases which are potentially treatable with such a medicament, may include keratitis and/or encephalitis, specifically granulomatous Acanthamoeba encephalitis (GAE) in humans.

In a sixth aspect of the present invention there is provided a method of treating a subject suffering from or susceptible to a disease caused and/or contributed to by the Amoebida, in particular Acanthamoeba, specifically for example Acanthamoeba castellanii and Acanthamoeba polyphaga, said method comprising the step of; a) administering to a subject in need thereof a therapeutically effective amount of a compound capable of modulating, preferably inhibiting, the histidine biosynthesis pathway, and/or the methionine biosynthesis pathway and/or a pathway branching from the histidine biosynthesis pathway and/or a pathway branching from the methionine biosynthesis pathway in the Amoebida.

When used for treating the above disorders, a compound capable of modulating the pathway and/or a pathway branching from the histidine biosynthetic pathway and/or the methionine biosynthesis pathway in the Amoebida may be administered in a variety of dosage forms. Thus, for example they can be administered as aqueous or oily suspensions, or in solution or any other suitable form.

When administered separately or sequentially, modulators, for example inhibitors, as described herein may be administered within any suitable time period, e.g. within 1, 2, 3, 6, 12, 24, 48 or 72 hours of each other. In preferred embodiments, they are administered within 6, preferably within 2, more preferably within 1, most preferably within 20 minutes of each other.

Whatever the compound used in a method of medical treatment of the present invention, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e. g. decisions on dosage etc, is within the responsibility of general practitioners and other medical doctors.

A compound capable of modulating the pathway and/or a pathway branching from the histidine biosynthetic pathway and/or the methionine biosynthesis pathway in the Amoebida, for example a peptide, polypeptide, nucleic acid modulator, small molecule, substance or composition may be administered alone or in combination with other treatments, either simultaneously or sequentially dependent upon the condition to be treated. For example, a compound capable of inhibiting the histidine pathway and a compound capable of inhibiting the methionine biosynthesis pathway may be provided simultaneously or sequentially to provide a synergistic effect. Alternatively, a compound capable of inhibiting the histidine and/or methionine biosynthesis pathway may be provided in simultaneous or sequential combination with a modulator, for example an inhibitor of the folate or shikimate pathway to provide a synergistic effect.

In a seventh aspect of the present invention there is provided a pharmaceutical formulation comprising a compound capable of modulating the histidine biosynthesis pathway, and/or the methionine biosynthesis pathway and/or a pathway branching from the histidine biosynthesis pathway and/or a pathway branching from the methionine biosynthesis pathway for use in treating keratitis caused or contributed to by a member of the order Amoebida, in association with a pharmaceutically acceptable carrier or diluent. In suitable embodiments, a compound capable of inhibiting the histidine biosynthetic pathway is provided in combination with an inhibitor of the methionine pathway to provide a synergistic effect for use in treating keratitis.

In embodiments a pharmaceutical formulation of the invention may be formulated, for example, in a form suitable for topical administration. For example the formulation for topical administration may be presented as a solution or a suspension in an aqueous or non-aqueous liquid, or as an oil-in-water liquid emulsion. In particular embodiments, the pharmaceutical formulation of the invention may be formulated such that it can be applied to the eye such as an eye care product which may include rewetting drops and artificial tears.

Conveniently the pharmaceutical formulation may be packaged within a receptacle to allow direct application to the eye. For example the pharmaceutical formulation may be packaged within a receptacle capable of delivering a predetermined volume of the formulation to the site of infection, for example the eye.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration. Preservatives, stabilisers, buffers, antioxidants and/or other additives may be included, as required.

The compounds for use in the invention may be administered in a localised manner to a desired site or may be delivered in a manner in which it targets the protozoa. Targeting therapies may be used to deliver the compounds of the invention more specifically to protozoa, by the use of targeting systems such as antibody or cell specific ligands. Targeting may be desirable for a variety of reasons, for example if the compound would otherwise require too high a dosage.

Instead of administering such substances directly, they may be produced in the protozoa by expression from an encoding nucleic acid introduced into the protozoa, e. g. from a vector. The vector may be targeted to the protozoa to be treated, or it may contain regulatory elements which are switched on more or less selectively by the protozoa. Nucleic acid encoding the compounds of the invention may thus be used in methods of gene therapy, for instance in treatment of individuals, e. g. with the aim of preventing or curing (wholly or partially) a disorder.

In an eighth aspect of the present invention there is provided a composition for cleaning contact lenses and/or storing contact lenses, said composition comprising a compound capable of modulating the histidine biosynthesis pathway, and/or the methionine biosynthesis pathway and/or a pathway branching from the histidine biosynthesis pathway and/or a pathway branching from the methionine biosynthesis pathway.

Such a composition may advantageously reduce infection caused and/or contributed to by members of the order Amoebida, for example Acanthamoeba castellanii and Acanthamoeba polyphaga.

In embodiments a composition for cleaning contact lenses and/or storing contact lenses may be provided as a solution. In particular embodiments, such a solution may comprise a neutral saline solution in combination with a compound for use in modulating the histidine biosynthesis pathway, and/or the methionine biosynthesis pathway and/or a pathway branching from the histidine biosynthesis pathway and/or a pathway branching from the methionine biosynthesis pathway. Suitably, in particular embodiments, a “one step” contact lens cleaning/storage solution may be provided with a compound for use in the histidine biosynthesis pathway, and/or the methionine biosynthesis pathway and/or a pathway branching from the histidine biosynthesis pathway and/or a pathway branching from the methionine biosynthesis pathway. For example a contact lens cleaning storage solution comprising a surfactant and/or combination of surfactants, selected from the non-ionic, anionic or amphoteric surfactants and a compound for use in modulating the histidine biosynthesis pathway, and/or the methionine biosynthesis pathway and/or a pathway branching from the histidine biosynthesis pathway and/or a pathway branching from the methionine biosynthesis pathway may be provided.

In a ninth aspect of the present invention there is provided a receptacle containing at least one contact lens and a composition suitable for cleaning contact lenses and/or storing contact lenses, said composition comprising a compound capable of modulating the histidine biosynthesis pathway, and/or the methionine biosynthesis pathway and/or a pathway branching from the histidine biosynthesis pathway and/or a pathway branching from the methionine biosynthesis pathway.

In embodiments the receptacle may be sealed such that the contents remain sterile until the receptacle is opened. In particular embodiments the receptacle may be resealable.

According to a tenth aspect of the present invention there is provided an in vitro method of detecting an Amoebida for example Acanthamoeba, in particular Acanthamoeba castellanii and Acanthamoeba polyphaga in a sample, said method comprising the steps of; a) providing a sample; and b) detecting the presence of any gene and/or protein of the histidine biosynthesis pathway, and/or the methionine biosynthesis pathway and/or a pathway branching from the histidine biosynthesis pathway and/or a pathway branching from the methionine biosynthesis pathway in the Amoebida.

In embodiments a sample may comprise cells or other bodily fluid derived from a subject suspected of being infected with an Amoebida. A bodily fluid may include, for example, whole blood, serum, plasma, cerebral spinal fluid (CSF), saliva or tears.

Any known technique in the art may be used to detect the presence of any gene and/or protein of the histidine biosynthesis pathway, and/or the methionine biosynthesis pathway and/or a pathway branching from the histidine biosynthesis pathway and/or a pathway branching from the methionine biosynthesis pathway in the Amoebida. In embodiments, either mRNA or protein can be measured as a means of determining up-or down regulation of the expression of a gene. Quantitative techniques are preferred. However, semi-quantitative or qualitative techniques can also be used. Suitable techniques for measuring gene products include, but are not limited to, SAGE analysis, DNA microarray analysis, Northern blot, Western blot, immunocytochemical analysis, ELISA, (PCR) or reverse transcriptase PCR (RT-PCR) or real-time PCR.

An oligonucleotide probe with binding specificity to a gene encoding a protein of the histidine biosynthesis pathway, and/or the methionine biosynthesis pathway and/or a pathway branching from the histidine biosynthesis pathway and/or a pathway branching from the methionine biosynthesis pathway in Amoebida may be used to identify such a gene. A suitable probe may consist of any portion of the genes of the histidine pathway or the methionine pathway described in the application. These may be produced via synthesis or through random priming techniques. In such a method, the oligonucleotide probe may be labelled for example with a chemiluminescent, fluorescent or radioactive label to allow visualisation/measurement of the probe or a secondary probe may be used to visualise/measure the oligonucleotide probe with binding specificity to a gene encoding a protein of the histidine biosynthesis pathway, and/or the methionine biosynthesis pathway and/or a pathway branching from the histidine biosynthesis pathway and/or a pathway branching from the methionine biosynthesis pathway in the Amoebida.

To detect a protein of the histidine biosynthesis pathway, and/or the methionine biosynthesis pathway and/or a pathway branching from the histidine biosynthesis pathway and/or a pathway branching from the methionine biosynthesis pathway in the Amoebida, methods as known to those in the art may be used, for example resolution of extracted proteins by means of native or denaturing SDS-polyacrylamide gel electrophoresis and then detection of said resolved proteins using for example silver stain, Coomassie blue stain, colloidal blue and colloidal gold stains.

Additionally or alternatively a protein may be detected, for example using an antibody specific for a particular protein or proteins. In such cases the antibody may be labelled, for example with a fluorescent, chemiluminesent or radioactive label such that binding of the antibody to the protein can be detected.

In an eleventh aspect of the present invention there is provided a method of identifying an agent which is capable of modulating, for example inhibiting, an enzyme or enzymes of the histidine biosynthesis pathway, and/or an agent capable of modulating, for example inhibiting, the methionine biosynthesis pathway and/or an agent capable of modulating, for example inhibiting, an enzyme or enzymes of any pathway branching from the histidine biosynthesis pathway and/or a pathway branching from the methionine biosynthesis pathway in the Amoebida, said method comprising the steps of;
(a) contacting a test agent with a system capable of expressing one or more enzymes of the histidine biosynthesis pathway, and/or the methionine biosynthesis pathway and/or a pathway branching from the histidine biosynthesis pathway and/or a pathway branching from the methionine biosynthesis pathway from an Amoebida species; and
(b) detecting any modulation of expression and/or activity of said one or more enzymes of the histidine biosynthesis pathway, and/or the methionine biosynthesis pathway and/or a pathway branching from the histidine biosynthesis pathway and/or a pathway branching from the methionine biosynthesis pathway,
(c) wherein modulation of expression and/or activity of said one or more enzymes of the histidine biosynthesis pathway, and/or the methionine biosynthesis pathway and/or a pathway branching from the histidine biosynthesis pathway and/or a pathway branching from the methionine biosynthesis pathway is indicative that the test agent is an agent which is capable of modulating an enzyme of the histidine biosynthesis pathway, and/or the methionine biosynthesis pathway and/or a pathway branching from the histidine biosynthesis pathway and/or a pathway branching from the methionine biosynthesis pathway in the Amoebida.

In particular embodiments, the step of detecting any modulation of expression and/or activity of said one or more enzymes of the histidine biosynthetic pathway and/or pathways branching from the histidine biosynthetic pathway may include detection of modulation of expression and/or activity of imidazoleglycerol-phosphate dehydratase (IGPD) polypeptide in Amoebida, the method comprising the steps of:
transforming a host cell with a nucleic acid fragment encoding an imidazoleglycerol-phosphate dehydratase from Amoebida, operably linked to a suitable regulatory sequence(s);
growing the transformed host cell under conditions that are suitable for expression of the imidazoleglycerol-phosphate dehydratase from Amoebida wherein expression of the imidazoleglycerol-phosphate dehydratase from Amoebida results in production of imidazoleglycerol-phosphate dehydratase in the transformed host cell;
optionally purifying the imidazoleglycerol-phosphate dehydratase expressed by the transformed host cell;
treating the imidazoleglycerol-phosphate dehydratase with a compound to be tested; and
comparing the activity of the imidazoleglycerol-phosphate dehydratase that has been treated with a test compound to the activity of an untreated imidazoleglycerol-phosphate dehydratase, and where differences are detected thereby selecting compounds with potential for modulatory, for example, inhibitory, activity.

As will be appreciated, this methodology may be applied to any of the enzymes of the histidine biosynthetic pathway or pathways branching from the histidine biosynthesis pathway, and/or the methionine biosynthesis pathway and/or a pathway branching from the histidine biosynthesis pathway and/or a pathway branching from the methionine biosynthesis pathway identified in Amoebida.

An agent may include a small organic molecule, an amino acid, a peptide, a protein or another suitable compound, for example a nucleic acid (DNA or RNA) or the like to be tested. As will be appreciated, such agents may be obtained from libraries (chemical or protein libraries).

It is considered that free-living Acanthamoeba may harbour pathogenic bacteria such as MRSA and Legionella. This is particularly dangerous in nosocomial environments as immunocompromised individuals are exposed to this.

Accordingly in a twelfth aspect of the present invention there is provided a compound which is capable of modulating the enzymes of the histidine biosynthesis pathway, and/or the methionine biosynthesis pathway and/or a pathway branching from the histidine biosynthesis pathway and/or a pathway branching from the methionine biosynthesis pathway in the Amoebida in combination with an anti-microbial compound suitable for use in treating pathogenic bacteria including MRSA and Legionella. In a further aspect of the present invention there is provided the use of a compound which is capable of modulating the enzymes of the histidine biosynthesis pathway, and/or the methionine biosynthesis pathway and/or a pathway branching from the histidine biosynthesis pathway and/or a pathway branching from the methionine biosynthesis pathway in the Amoebida for treating pathogenic bacteria including MRSA and Legionella. In a yet further aspect of the invention there is provided bactericidal, antibacterial, anti-infective, antimicrobial, sporicidal, disinfectant, antifungal, germicidal and antiviral agents comprising a compound which is capable of modulating the enzymes of the histidine biosynthesis pathway, and/or the methionine biosynthesis pathway and/or a pathway branching from the histidine biosynthesis pathway and/or a pathway branching from the methionine biosynthesis pathway in the Amoebida.

As indicated above, Acanthamoeba species can parasitise humans causing severe debilating eye disease individuals, for example Acanthamoeba keratitis, with infection being increased in contact lens wearers. It would be advantageous to produce contact lenses that inhibit the growth of Acanthamoeba and other bacteria or other microbes. Accordingly, there is provided an eye prosthesis, a contact lens, an eye prosthesis or contact lens storage container comprising a compound which is capable of modulating the enzymes of the histidine biosynthesis pathway, and/or the methionine biosynthesis pathway and/or a pathway branching from the histidine biosynthesis pathway and/or a pathway branching from the methionine biosynthesis pathway in the Amoebida. In a further aspect there is provided a method of preparing an antimicrobial lens or eye prosthesis comprising, (a) treating a contact lens or eye prosthesis, with a compound which is capable of modulating at least one of the enzymes of the histidine biosynthesis pathway, and/or the methionine biosynthesis pathway and/or a pathway branching from the histidine biosynthesis pathway and/or a pathway branching from the methionine biosynthesis pathway in the Amoebida and (b) treating the lens or eye prosthesis of step (a) to bind the compound to the surface of the contact lens or prosthesis. Suitably, in embodiments, binding of the compound may be provided for by a step of providing suitable functional groups on the surface of the contact lens or prosthesis.

Defined Terms

The term “inhibition” may be taken to mean, when compared to an enzyme which has not been contacted and/or exposed to a compound which might inhibit its activity, the rate at which an enzyme catalyses the production of a particular compound from a particular substrate is reduced.

The term “non-specific” may be taken to mean a compound, the effects of which are not restricted to a particular enzyme, or class of enzyme, but which generally affects the activity of substantially all enzymes. Typically non-specific inhibitors may function as a result of the fact they denature by means of a change in temperature, pH or the like. However, non-specific inhibitors may also irreversibly bind to and block the active site of an enzyme thus preventing it from functioning to catalyse the production of a particular compound.

The term “specific inhibitor” may be taken to refer to a compound which may inhibit a particular enzyme but which has no effect on the activity of another enzyme. Specific enzyme inhibitors may function by binding to a particular enzyme, for example at or around an active site of an enzyme causing the active site to be blocked or sterically hindering the approach of substrate to the active site. Alternatively, or additionally, an inhibitor may bind an enzyme at a region other than the active site and cause the enzyme to undergo a conformational change such that it can no longer receive and catalyse the conversion of a particular substrate.

The term “competitive inhibitor” may be taken to comprise compounds which are capable of occupying the active site of a particular enzyme resulting in the exclusion of the correct substrate such that the rate at which the enzyme catalyses a reaction is reduced. In particular embodiments such compounds may closely resemble the native (natural/correct) substrate of the enzyme.

The term “non-competitive inhibitor” may be taken to include those compounds which affect the rate at which an enzyme catalyses the production of a particular compound. In contrast to the competitive inhibitors, non-competitive inhibitors may interact with an enzyme at a position other that the active site of an enzyme. For example a non-competitive inhibitor may bind to an enzyme and induce a conformational change in the enzyme such that the binding site of the enzyme may no longer receive a substrate.

A “system capable of expressing one or more enzymes of the histidine biosynthetic pathway and/or pathways branching from the histidine biosynthetic pathway from an Amoebida species” may be a cell-free or a cell-based systems. The “system” may comprise one or more enzymes derived from the histidine biosynthetic pathway and/or a pathway branching from the histidine biosynthetic pathway. Additionally, the “system” may comprise a substrate or substrates with which said enzyme or enzymes can interact.

The term “modulation” may be taken to mean an increase or decrease in enzymatic activity and/or an increase or decrease in enzyme expression when compared to the activity or expression of the same enzyme which has not been exposed a test agent or compound.

Modulation of expression may be detected by, for example, creating a fusion protein comprising an enzyme of the histidine biosynthetic pathway and/or an enzyme of a pathway branching from the histidine biosynthetic pathway and a detectable tag, for example a chemiluminescent, bioluminescent, fluorescent tag, a His or GST tag or another suitable tag which may be detected by means of an antibody or similar. Suitably using such a methodology, an increase in the expression of the enzyme would result in more tag being detected. The level of expression of an enzyme or enzymes may also be determined by means of quantifying the amount of specific mRNA present for example using quantitative real-time PCR, agarose gel electrophoresis and/or Northern/Southern blotting.

Throughout the specification, unless the context demands otherwise, the terms ‘comprise’ or ‘include’, or variations such as ‘comprises’ or ‘comprising’, ‘includes’ or ‘including’ will be understood to imply the includes of a stated integer or group of integers, but not the exclusion of any other integer or group of integers.

In embodiments of the invention, for all aspects of the invention disclosed herein, the modulator of the biosynthesis pathway can be an inhibitor.

Preferred features and embodiments of each aspect of the invention are as for each of the other aspects mutatis mutandis unless context demands otherwise.

### DETAILED DESCRIPTION OF THE INVENTION

The present invention will now be described by way of example only with reference to the figures which show:

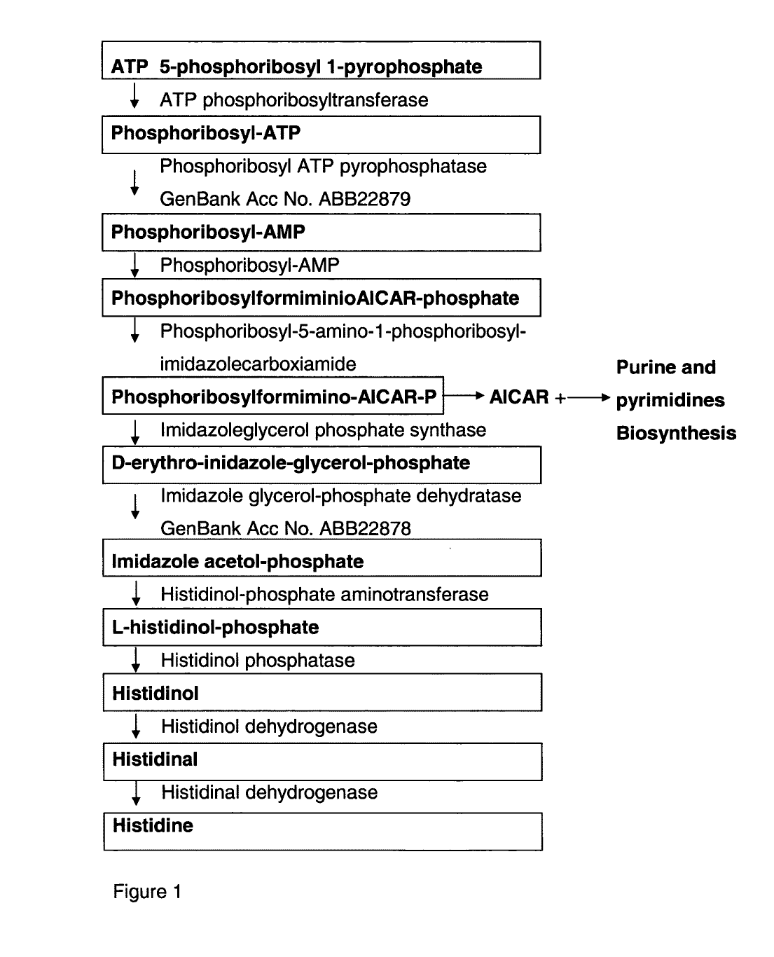

FIG. 1 illustrates the histidine biosynthesis pathway consisting of 10 enzymatic reactions;

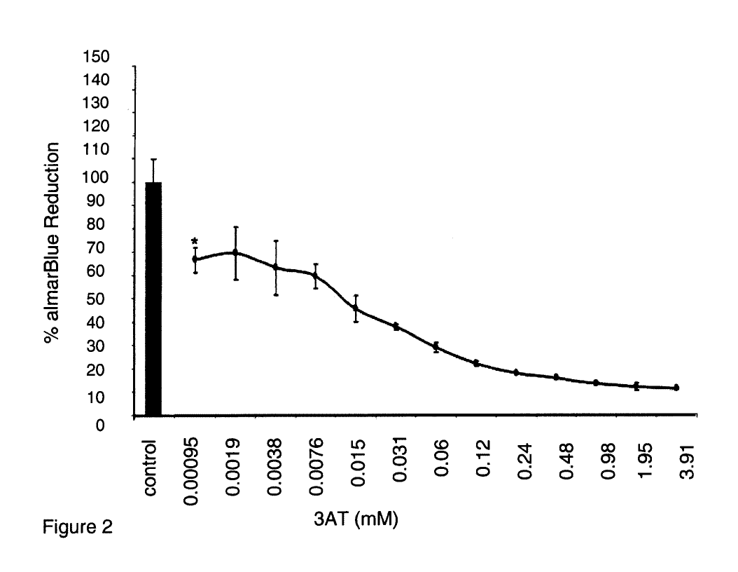

FIG. 2 illustrates Acanthamoeba growth inhibition in the absence of histidine, wherein Acanthamoeba growth is inhibited in a dose dependent manner—the IC₅₀ is between 0.015 mM and 0.031 mM—the X axis represents the dose of 3-AT in mM and the Y-axis is the percentage of alamarBlue reduction, which is directly proportional to mitochondrial activity;

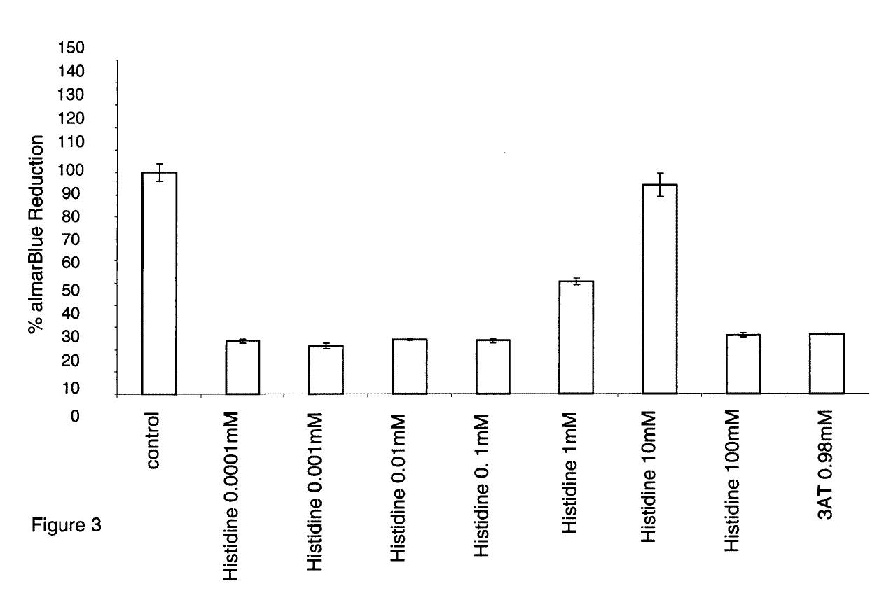

FIG. 3 illustrates Acanthamoeba growth inhibition is ablated in the presence of histidine wherein the addition of 10 mM histidine can allow Acanthamoeba to grow in the presence of 0.98 mM (IC₂₀) 3-AT—the x-axis represents the quantity of histidine, whereas the y-axis represents the percentage of alamarBlue reduction and 100 mM histidine was toxic to Acanthamoeba, but at 10 mM histidine significantly (p<0.05) ablated the effect of 3AT;

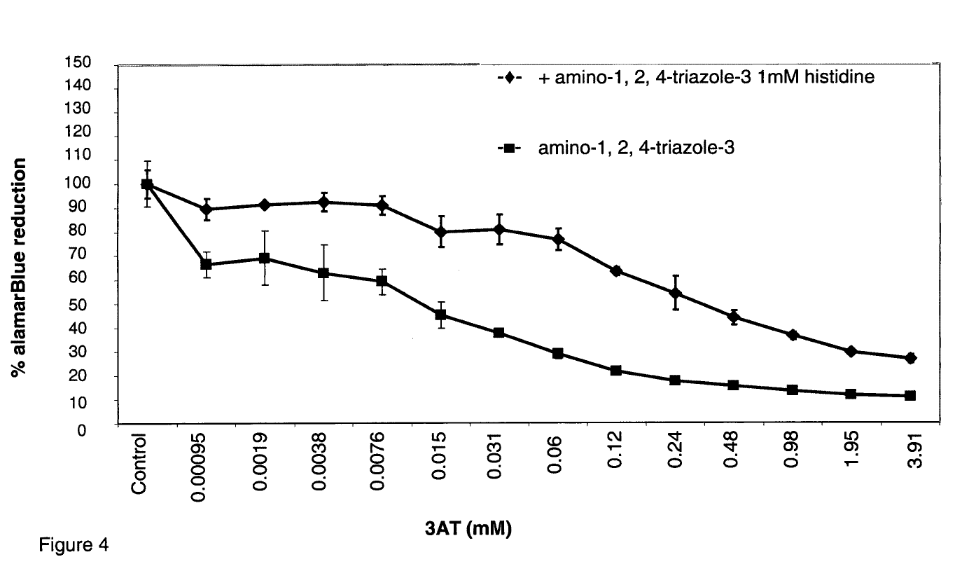

FIG. 4 illustrates a reduction of alamarBlue over a range of concentrations of 3AT wherein 3AT (▪) significantly (p<0.05) inhibited A. castellanii growth, compared with untreated control cultures, in a dose dependent manner with concentrations of 0.00095 mM or above. In the presence of 1 mM Histidine (♦) this was reduced and 0.06 mM 3AT or above was required in order to significantly (p<0.05) inhibited A. castellanii growth;

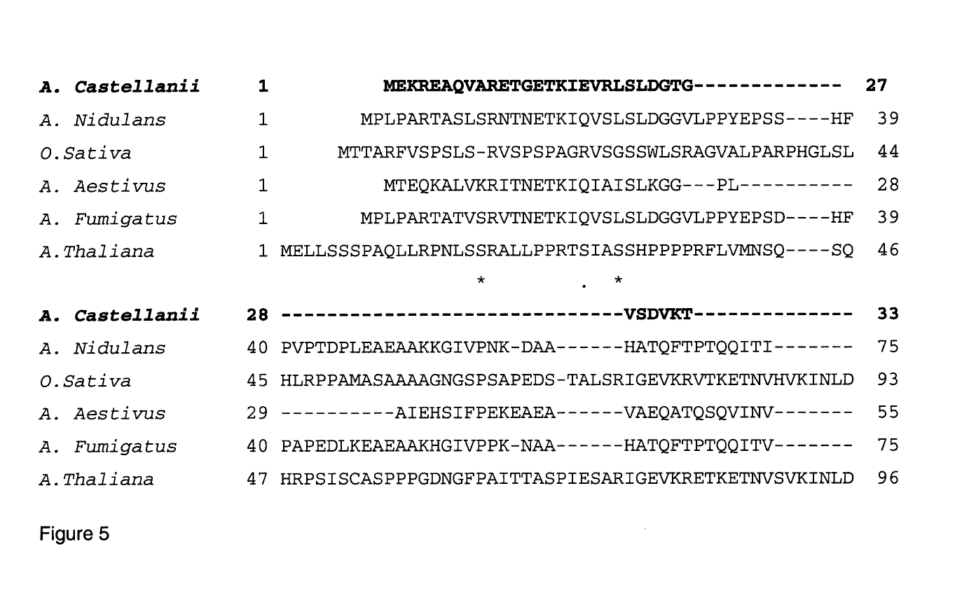

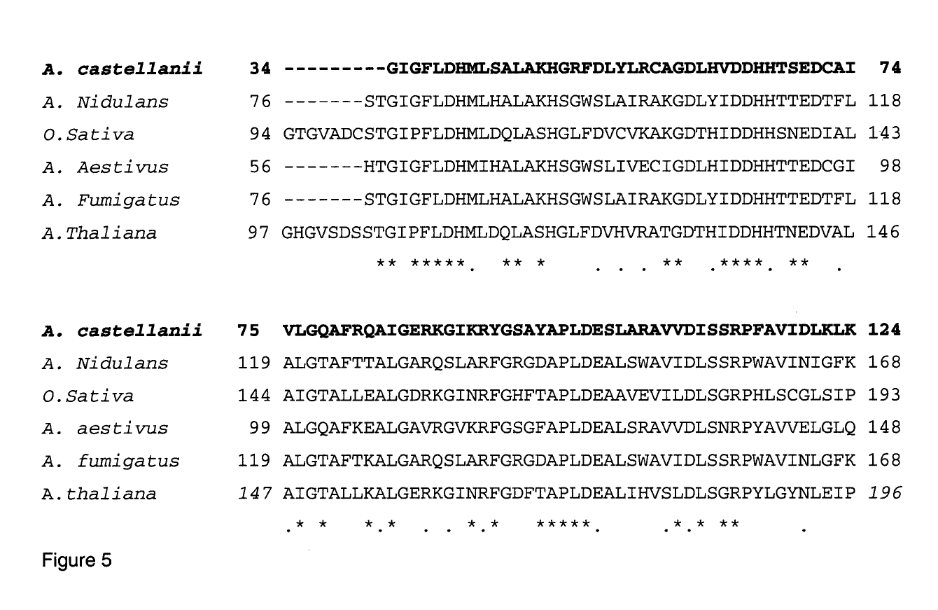

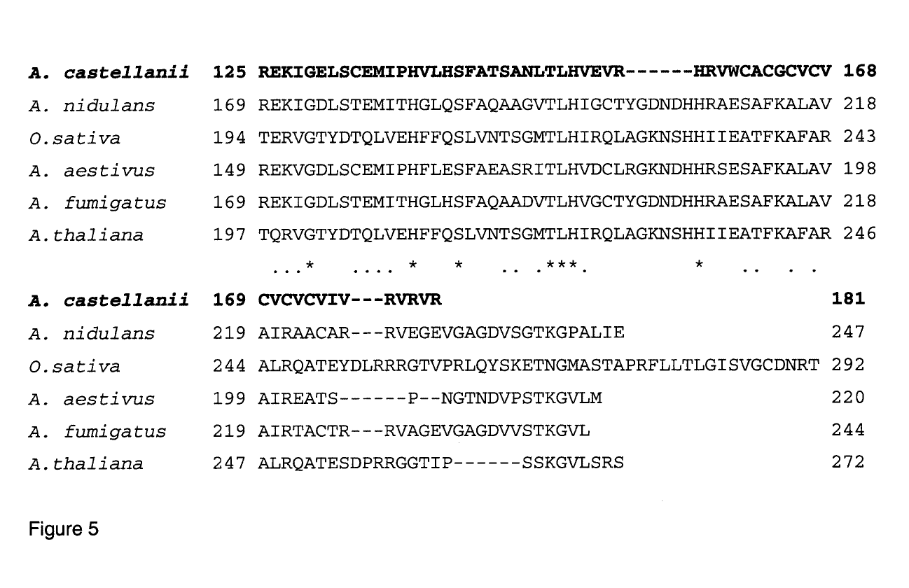

FIG. 5 illustrates an alignment of IGPD gene of Acanthamoeba;

FIG. 6 illustrates the cobalamin-independent methionine biosynthesis pathway and its enzymatic reactions.

### EXAMPLE 1

The inventors have cloned and sequenced the sixth enzyme in the histidine biosynthetic pathway, imidazole glycerol-phosphate dehydratase (IGPD) (EC 4.2.1.19), from A. castellanii (see FIG. 1). This enzyme is the molecular target for 3-amino-1,2,4-triazole (3AT), a commonly used commercially available herbicide

The imidazoleglycerol-phosphate dehydratase (IGPD) gene (hisB) of Acanthamoeba can be obtained according to the following protocol.

In a total volume of 25 μl, 1 μl, cDNA (derived from Acanthamoeba grown in the absence of histidine) was added to 1 μl 10 mM dNTP mixture, 2 mM MgSO4, 25 pmol of forward primer (5′ GAAAAGAGGGAGGCACAGGT 3′) (SEQ ID NO 1) and 25 pmol of reverse primer (5′ ATCACTCGAGTACGCCCTTGG 3′) (SEQ ID NO 2) in the presence of 1 Unit of Platinum Taq High Fidelity and 10× High Fidelity Buffer. The thermo cycling conditions consist of an initial denaturation at 95° C. for 3 minutes, followed by 35 cycles of denaturation at 95° C. for 30 seconds, annealing at 62° C. and extension at 72° C. A final extension step is at 72° C. for 10 minutes. These thermocycling conditions were also used to obtain the fragments of the other genes of enzymes belonging to the histidine biosynthesis pathway.

Imidazoleglycerol-phosphate dehydratase (IGPD) gene (hisB) of Acanthamoeba

(SEQ ID NO 3)
GAAAAGGTAAAGGAAATTAAACCATGAAAAGAAAAAGGACAAGAAATAT
ATAATTGCAAGAGAGATGGAAAAGAGGGAGGCACAGGTGGCGCGAGAGA
CTGGGGAGACCAAGATCGAGGTGCGTCTGTCGCTCGATGGCACGGGCGT
GTCCGATGTGAAGACCGGCATCGGCTTCCTGGACCACATGCTGTCCGCC
CTCGCCAAGCATGGTCGCTTCGATCTCTATCTCCGATGCGCGGGCGATC
TCCACGTAGACGACCACCACACCTCCGAGGACTGCGCGATCGTGCTGGG
CCAAGCCTTCCGCCAGGCTATTGGTGAGCGTAAGGGCATCAAGCGTTAT
GGGTCGGCCTACGCGCCGCTGGACGAGAGCCTGGCGCGCGCCGTGGTGG
ACATCTCGTCGCGCCCGTTCGCCGTGATCGACCTCAAGCTCAAGCGGGA
GAAGATCGGAGAGCTCTCGTGCGAGATGATCCCTCACGTCCTTCACTCC
TTCGCCACCAGCGCCAACCTCACACTTCACGTTGAGGTCCTGTACGGTG
CGAATGACCACCACAAGGCCGAATCGGCATTCAAGGCCACTGCGCTGGC
CCTGCGCGAGGCTGTGACCAAAGACGGTCCCGCCGACGCCGTGCCCAGC
ACCAAGGGCGTACTCGAGTGAT

### EXAMPLE 2

As illustrated in FIGS. 2 and 3, the inventors have determined that Acanthamoeba can grow in the absence of histidine and that 3-amino triazole, an inhibitor of IGPD, the sixth enzyme of the histidine biosynthetic pathway inhibits growth of Acanthamoeba. Furthermore, inhibition was ablated in a dose dependent manner by the addition of histidine to the medium. This demonstrates that 3AT acts specifically to inhibit IGPD and that the histidine biosynthesis pathway in A. castellanii. This provides the first evidence of the histidine biosynthesis pathway in a protozoan and demonstrates its potential as a target for therapeutic intervention during Acanthamoeba infection.

### EXAMPLE 3

The inventors have further identified portions of genes that transcribe enzymes belonging to the histidine pathway providing further support that the histidine biosynthetic pathway is present in Acanthamoeba.

ATP Phosphoribosyltransferase Partial Genomic Sequence

(SEQ ID NO 4)
GATCGAGGGCAGGCCCGCGATGACCGACTCCAGGCACGACTTCTCGACG
TTGAACTCGATCAGGGCCTTCTCGCGGGCGTTGAGCGCCGACTGCAGGA
GGATCACGAACTCGTCCAACGCCGCGTGCTTACGCGGGTCCTCCAGCGC
GGTGCGGTTGATGCAGAGGAAGGTGGAGGACTTGGTGATCTCATCGACG
ATGGCCAGCTGGTTGGCCCGCAGGGTGGTGC

Phosphoribosyl ATP Pyrophosphatase Partial Coding Sequence

(SEQ ID NO 5)
GAGCTCGCCGATGCTGAAGGCAAGGAGCACATCGCCAACGAGGCTGCCG
ATCTTCTCTACTTTGCGCTGGTGGCCTGCACCAAGGCCGGCGTCACCCT
GTCCGACGTCGAGCACGTCCTT

Phosphoribosyl-5-amino-1-Phosphoribosyl-Imidazolecarboxamide Isomerase Partial Genomic Sequence

(SEQ ID NO 6)
GCCGTGCCTGGCGTCCAGCGCGACCACCACGCGCTCCCTGGGCAGCTGC
CGCAGGAAGTCGGGCTTGGCGGCCGTGCCGATCCCCACCTTGGCAGCGC
CAGCGTTTAGCCAATCAC

Imidazoleglycerol-Phosphate Dehydratase, Partial Coding Sequence

(SEQ ID NO 7)
ATGGAAAAGAGGGAGGCACAGGTGGCACGAGAGACGGGGGAGACCAAGA
TCGAGGTGCGCCTGTCGCTCGACGGCACGGGCGTGTCCGATGTGAAGAC
CGGCATCGGCTTCCTGGACCACATGCTGTCCGCCCTCGCCAAGCACGGT
CGCTTCGATCTCTATCTCCGATGCGCGGGCGATCTCCACGTCGACGACC
ACCACACCTCCGAGGACTGCGCGATCGTGCTGGGCCAAGCCTTCCGCCA
GGCCATCGGTGAGCGTAAGGGCATCAAGCGGTACGGGTCGGCCTACGCG
CCGCTGGACGAGAGCCTGGCGCGCGCCGTGGTGGACATCTCGTCTCCCC
CGTCTCTCGCGAATCT

Histidinol Dehydrogenase Partial Genomic Sequence

(SEQ ID NO 8)
ACATGCTCGCCGGTCCCTCCGAGTGCCTCGTGCTAGCCGACGGCACGGC
CCACCCGGACGTCGTGGCGGCCGACCTGCTGGCCCAGGCCGAGCACGAC
GTGGCGGCCGTGCCCATCTTGGTGGCCTACGACGAGGAGACCGTGCGCC
GGGTCGAACAGGAGGTCGAGGCTCAGCTCGCCACCCTGCCCACCGCTGC
CATCGCCCGCGCCGCCTTCTCCAACGGTTTTGCTGTGATTGTCGCGAAC
GAAGAGGAGGCCATCACGGTGTGCGACAAGCTCGCGCCCGAGCACTTGG
AGG

### EXAMPLE 4

The inventors have also determined portions of genes that transcribe enzymes belonging to the methionine pathway which can synthesise methionine from cysteine in a cobalamin-independent manner (see FIG. 6).

The inventors have further established that Acanthamoeba castellanii possesses this metabolic pathway as they have identified that that it expresses two genes that transcribe enzymes involved in methionine biosynthesis, namely, methionine synthase and cystathione gamma synthase.

Currently, there are no known inhibitors for cobalamin-independent methionine synthase but studies concentrating on cobalamin-independent methionine synthases from fungal species are expected to identify plausible candidates.

Inhibitors of cystathionine gamma synthase include dl-E-2-amino-5-phosphono-3-pentenoic acid, 3-(phosphonomethyl) pyridine-2-carboxylic acid and 5-carboxymethylthio-3-(3′-chlorophenyl)-1,2,4-oxadiazol.

Using the protocol and thermocycling conditions of Example 1, fragments of the genes of methionine synthase and cystathione gamma synthase, belonging to the methionine biosyntheis pathway, were obtained. The reverse and forward primers for use therein are a shown in the table 1 below.

TABLE 1

| Enzyme | Abbrvn | Forward | Reverse |
|---|---|---|---|
| cystathionine | CGS | 5′-ATTTGGATGGA | 5′CTTGGTCACC |
| gamma- | | GTCGCCGACC-3′ | GAGTGGACG-3′ |
| synthase | | (SEQ ID NO 9) | (SEQ ID NO 10) |
| methionine | MS1 | 5′-AAGCGCGAGGG | 5′-GTCGCCACGG |
| synthase | | GATGAGGCCGAG-3′ | GCACCATCGGC-3′ |
| | | (SEQ ID NO 11) | (SEQ ID NO 12) |
| | MS2 | 5′-TGCCAGACGT | 5′-CCAGGGCGC |
| | | TGCGGGCATC-3′ | TGCACATGAC-3′ |
| | | (SEQ ID NO 13) | (SEQ ID NO 14) |
| | MS5 | 5′-GACTCGTACTC | 5′-ATCGACGCG |
| | | (SEQ ID NO 15) | CTCGATGCCG-3' |
| | | GGCCCGCAGG-3′ | (SEQ ID NO 16) |

AcCGS—Cystathionine Gamma-Synthase Partial Genomic Sequence

(SEQ ID NO 17)
atttggatggagtcgccgaccaacccgaccctcaagctcaccgacatcc
gcaaggtggcggaggtggccaagaagaggaacatcctgctggtcgtgga
cgacaccttcatgtcgccctacttccagaaccccctcgacctcggcgcc
gacatcgtcgtccactcggtgaccaag

Using the above protocol, different sections of the methionine synthase gene were determined as described below.

MS1—Methionine Synthase Partial Genomic Sequence

(SEQ ID NO 18)
gttgatgacatcatcagcaagctcgccgccatccgcgcgtcgttcgagg
gcaagctggtggtccagccctcggcgtcgcttcagcacgtgccctacga
tgccaccgtggagaccaagctgccctcggccctgcgccaggtccttgcc
ttcgccttccagaagctcaaggaggtcgacttcatcgctag

MS2—Methionine Synthase Partial Genomic Sequence

(SEQ ID NO 19)
gtcgccacgggcaccatcggcttcccgcgtgtgggcaaggaccgagagc
ttaagaaggcgctcgagtcctactgggccggcaccaccgaccgtaccca
tttgctcggcgtggccgcccaggtcgagaaggccaacctcctcgcccag
gtcgacgccggcatcgagcgcatcggcgtaggcagcttcagcctctacg
accaggtcctcgactgggccttccgcctcggcctcatcccctcgcgctt

MS5—Methionine Synthase Partial Genomic Sequence

(SEQ ID NO 20)
atcgacgcgctcgatgccgatgtgatctcgatcgagaactcgcgtaaca
acgacaagacgattaaggacctcaccgcctacggctacgagcgcgacat
cggccccggcgtgtacgacatccacagcccggtggtgcccaccgtcgac
gagattgtcgagaagctgcgcctcttcctcaagcacctccacccgtcgc
gcgtcgtggtcaaccccgactgcggcctcaagacccgccagtggaagga
ggtcgtgccgtcgctgcgcaacatggtcaccgccgccagcatcctgcgg
gccgagtacgagtc

### EXAMPLE 5

The inventors have found that inhibitors of the histidine and/or methionine pathways are useful in eye care products including artificial tears and rewetting drops. In such preparations the inhibitors can generally function as preservative(s) and so prolong the product shelf life by preventing Acanthamoeba contamination.

A typical eye care product formulation may include water and salts with one or more of: carboxymethyl cellulose, hydroxypropyl methycellulose, hydroxypropyl cellulose, glycerin, propylene glycol, polyethylene glycol (PEG 400) and HP-Guar. Important aspects of such formulations are lubrication and osmoprotection and the formulations must be compatible with contact lens wear as they would be considered as in eye contact lens solutions.

Although the invention has been particularly shown and described with reference to particular examples, it will be understood by those skilled in the art that various changes in the form and details may be made therein without departing from the scope of the present invention

1. Use of a compound capable of modulating the histidine biosynthesis pathway, and/or the methionine biosynthesis pathway and/or a pathway branching from the histidine biosynthesis pathway and/or methionine biosynthesis pathway in the control of growth of at least one organism of the order of Amoebida.

2. Use as claimed in claim 1 in the control of growth of at least one organism of the order of Amoebida, wherein said compound is capable of modulating the histidine biosynthesis pathway in said organism.

3. Use as claimed in claim 1 in the control of growth of at least one organism of the order of Amoebida, wherein said compound is capable of modulating the methionine biosynthesis pathway in said organism.

4. Use as claimed in claim 1, wherein the at least one organism is of the family of Acanthamoebidae.

5. Use as claimed in claim 4, wherein the at least one organism is of the genus Acanthamoeba.

6. Use as claimed in claim 5, wherein the at least one organism is Acanthamoeba castellanii or Acanthamoeba polyphaga.

7. Use as claimed in claim 1, wherein the compound is selected from
i) an inhibitor of at least one enzyme of the histidine biosynthesis pathway or the methionine biosynthesis pathway,
ii) an inhibitor of at least one enzyme of a pathway branching from the histidine biosynthesis pathway or the methionine biosynthesis pathway,
iii) an inhibitor of the expression of an enzyme of the histidine biosynthesis pathway or the methionine biosynthesis pathway, or
iv) an inhibitor of the expression of an enzyme of a pathway branching from the histidine biosynthesis pathway or methionine biosynthesis pathway.

8. Use as claimed in claim 7, wherein the compound is an oligonucleotide sequence which binds to a nucleic acid sequence encoding at least one enzyme of the histidine biosynthesis pathway,
a nucleic acid sequence encoding at least one enzyme of the methionine biosynthesis pathway,
a nucleic acid sequence encoding at least one enzyme of a pathway branching from the histidine biosynthesis pathway,
a nucleic acid sequence encoding at least one enzyme branching from the methionine biosynthesis pathway, or
a nucleic acid sequence which controls the expression of a nucleic acid sequence encoding an enzyme of the histidine or methionine biosynthesis pathway.

9. Use as claimed in claim 8, wherein the oligonucleotide sequence is RNAi, siRNA or at least one antisense oligonucleotide.

10. Use as claimed in claim 1, wherein the compound is a small organic molecule.

11. Use as claimed in claim 10, wherein the small organic molecule is an analogue of at least one substrate of at least one of the enzymes utilised in the histidine biosynthesis pathway, or an analogue of at least one substrate of at least one of the enzymes utilised in the methionine biosynthesis pathway or of an enzyme of a pathway branching from the histidine biosynthesis pathway or methionine biosynthesis pathway.

12. Use as claimed in claim 10, wherein the small molecule is 3-amino-1,2,4-triazole (3AT).

13. Use as claimed in claim 1, wherein the compound is provided in at least one of; a contact lens solution, a coating on a surgical instrument, a coating on prosthesis and/or in medicated soap.

14. A composition for use in modulating the histidine biosynthesis pathway and/or a pathway branching from the histidine biosynthesis pathway in Amoebida and/or the methionine biosynthesis pathway and/or a pathway branching from the methionine biosynthesis pathway in Amoebida, wherein the composition comprises a first compound capable of modulating the activity and/or expression of
i) at least one enzyme of the histidine biosynthesis pathway and/or the methionine biosynthesis pathway
ii) at least one enzyme of a pathway branching from the histidine and/or methionine biosynthesis pathway, and at least a second compound wherein said second compound is capable of modulating a further biosynthesis pathway present in Amoebida.

15. The composition as claimed in claim 14, wherein the second compound is capable of modulating an enzyme of the shikimate or the folate pathway.

16. The composition as claimed in claim 14, wherein the second compound is selected from the group consisting of sulphonamide, pyrimethamine, proguanil, cycloguanyl or trimethoprim or a derivative thereof, thazine, PS-15, hexamidine, biguanide or chlorhexidine digluconate.

17. The composition as claimed in claim 14, wherein the first compound is an inhibitor of
i) at least one enzyme of the histidine biosynthesis pathway and/or the methionine biosynthesis pathway and/or
ii) at least one enzyme of a pathway branching from the histidine and/or methionine biosynthesis pathway.

18. A compound being capable of modulating, preferably inhibiting, the histidine biosynthesis pathway, and/or the methionine biosynthesis pathway, and/or a pathway branching from the histidine biosynthesis pathway and/or a pathway branching from the methionine biosynthesis pathway for the treatment of a disease caused and/or contributed to by at least one organism of the order of Amoebida.

19. A composition as claimed in claim 14 for the treatment of a disease caused and/or contributed to by the Amoebida.

20. A compound or composition as claimed in claim 18, wherein the disease is keratitis or encephalitis.

21. A method of treating a subject suffering from or susceptible to a disease caused and/or contributed to by a Amoebida, said method comprising the step of;
administering to a subject in need thereof
(a) a therapeutically effective amount of a compound capable of modulating, preferably inhibiting, the histidine biosynthesis pathway, and/or
(b) a therapeutically effective amount of a compound capable of modulating, preferably inhibiting, the methionine biosynthesis pathway, and/or
(c) a therapeutically effective amount of a compound capable of modulating, preferably inhibiting, a pathway branching from the histidine or methionine biosynthesis pathway in the Amoebida.

22. The method as claimed in claim 21, wherein the Amoebida is Acanthamoeba castellanii or Acanthamoeba polyphaga.

23. A pharmaceutical formulation comprising a composition as claimed in claim 14.

24. A composition for cleaning contact lenses and/or storing contact lenses comprising a compound capable of modulating, preferably inhibiting, the histidine biosynthesis pathway and/or a pathway branching from the histidine biosynthesis pathway and an anti-bacterial agent and/or the methionine biosynthesis pathway and/or a pathway branching from the methionine biosynthesis pathway.

25. A receptacle for cleaning and/or storing contact lenses, said receptacle containing at least one contact lens and a composition as claimed in claim 24.

26. An in vitro method of detecting an Amoebida in a sample, said method comprising the steps of;
a) providing a sample; and
b) detecting the presence of
i) a gene and/or protein of the histidine biosynthesis pathway,
ii) a gene and/or protein of the methionine biosynthesis pathway,
iii) a gene and/or protein of a pathway branching from the histidine biosynthesis pathway or
iv) a gene and/or protein of a pathway branching from the methionine biosynthesis pathway, wherein said gene is a gene encoded by Amoebida and said protein is a protein encoded by Amoebida.

27. The in vitro method as claimed in claim 26, wherein the sample comprises cells and/or bodily fluid derived from a subject suspected of being infected with an Amoebida.

28. A method of identifying an agent being capable of modulating an enzyme or enzymes of the histidine and/or methionine biosynthesis pathways and/or an enzyme or enzymes of any pathway branching from the histidine or methionine biosynthesis pathways in the Amoebida, said method comprising the steps of;
(a) contacting a test agent with a system capable of expressing one or more enzymes of the histidine and/or methionine biosynthesis pathways and/or pathways branching from the histidine and/or methionine biosynthesis pathways from an Amoebida species; and
(b) detecting a modulation of expression and/or activity of said one or more enzymes of the histidine and/or methionine biosynthesis pathways and/or pathways branching from the histidine and/or methionine biosynthesis pathways, wherein detection of said modulation of expression and/or activity of said one or more enzymes in step (b) is indicative that the test agent is an agent which is capable of modulating an enzyme of the histidine and/or methionine biosynthesis pathways and/or an enzyme of any pathway branching from the histidine and/or methionine biosynthesis pathways in the Amoebida.

29. The method as claimed in claim 28, wherein step (b) includes detection of modulation of expression and/or activity of imidazoleglycerolphosphate dehydratase (IGPD) polypeptide in Amoebida comprising the steps of:
a) transforming a host cell with a nucleic acid fragment encoding an imidazoleglycerol-phosphate dehydratase from Amoebida, operably linked to suitable regulatory sequences;
b) growing the transformed host cell under conditions that are suitable for expression of the imidazoleglycerol-phosphate dehydratase from Amoebida, wherein expression of the imidazoleglycerol-phosphate dehydratase from Amoebida results in production of imidazoleglycerol-phosphate dehydratase in the transformed host cell;
c) treating the imidazoleglycerol-phosphate dehydratase with a compound to be tested; and
d) comparing the activity of the imidazoleglycerol-phosphate dehydratase that has been treated with a test compound to the activity of an untreated imidazoleglycerol-phosphate dehydratase.

30. The method as claimed in claim 29, further comprising the step of purifying the imidazoleglycerol-phosphate dehydratase expressed by the transformed host cell preceding step c).

31. A composition comprising a compound capable of modulating, preferably inhibiting, the histidine and/or methionine biosynthesis pathways or a pathway branching from the histidine and/or methionine biosynthesis pathways, and a second antimicrobial compound wherein the antimicrobial compound is effective in reducing the growth of MRSA or Legionella.

32. Use of the composition comprising a compound capable of modulating, preferably inhibiting, the histidine and/or methionine biosynthesis pathways and/or a pathway branching from the histidine and/or methionine biosynthesis pathways, and a second antimicrobial compound wherein the antimicrobial compound is effective in reducing the growth of MRSA or Legionella for the treatment of MRSA or Legionella.

33. A contact lens or eye prosthesis comprising a compound capable of modulating, preferably inhibiting, at least one enzyme of the histidine and/or methionine biosynthesis pathways and/or of a pathway branching from the histidine and/or methionine biosynthesis pathways in the Amoebida.

34. A method of preparing a contact lens or eye prosthesis as claimed in claim 33, comprising the steps of:
(a) treating a contact lens or eye prosthesis with a compound capable of modulating, preferably inhibiting, at least one of the enzymes of the histidine and/or methionine biosynthesis pathways and/or at least one of the enzymes of a pathway branching from the histidine and/or biosynthesis pathways in the Amoebida; and
(b) treating the lens or eye prosthesis of step (a) with binder to bind the compound to the surface of the contact lens or eye prosthesis.


SEQUENCE LISTING (26 sequences)

SEQ ID NO: 1 - DNA, 20 bp, Artificial Sequence
  oligonucleotide (forward primer)
gaaaagagggaggcacaggt

SEQ ID NO: 2 - DNA, 21 bp, Artificial Sequence
  oligonucleotide (reverse primer)
atcactcgagtacgcccttgg

SEQ ID NO: 3 - DNA, 659 bp, Acanthamoeba castellanii
gaaaaggtaaaggaaattaaaccatgaaaagaaaaaggacaagaaatatataattgcaagagagatggaaaagagggaggcacaggtggcgcgagagactggggagaccaagatcgaggtgcgtctgtcgctcgatggcacgggcgtgtccgatgtgaagaccggcatcggcttcctggaccacatgctgtccgccctcgccaagcatggtcgcttcgatctctatctccgatgcgcgggcgatctccacgtagacgaccaccacacctccgaggactgcgcgatcgtgctgggccaagccttccgccaggctattggtgagcgtaagggcatcaagcgttatgggtcggcctacgcgccgctggacgagagcctggcgcgcgccgtggtggacatctcgtcgcgcccgttcgccgtgatcgacctcaagctcaagcgggagaagatcggagagctctcgtgcgagatgatccctcacgtccttcactccttcgccaccagcgccaacctcacacttcacgttgaggtcctgtacggtgcgaatgaccaccacaaggccgaatcggcattcaaggccactgcgctggccctgcgcgaggctgtgaccaaagacggtcccgccgacgccgtgcccagcaccaagggcgtactcgagtgat

SEQ ID NO: 4 - DNA, 227 bp, Acanthamoeba castellanii
gatcgagggcaggcccgcgatgaccgactccaggcacgacttctcgacgttgaactcgatcagggccttctcgcgggcgttgagcgccgactgcaggaggatcacgaactcgtccaacgccgcgtgcttacgcgggtcctccagcgcggtgcggttgatgcagaggaaggtggaggacttggtgatctcatcgacgatggccagctggttggcccgcagggtggtgc

SEQ ID NO: 5 - DNA, 120 bp, Acanthamoeba castellanii
gagctcgccgatgctgaaggcaaggagcacatcgccaacgaggctgccgatcttctctactttgcgctggtggcctgcaccaaggccggcgtcaccctgtccgacgtcgagcacgtcctt

SEQ ID NO: 6 - DNA, 116 bp, Acanthamoeba castellanii
gccgtgcctggcgtccagcgcgaccaccacgcgctccctgggcagctgccgcaggaagtcgggcttggcggccgtgccgatccccaccttggcagcgccagcgtttagccaatcac

SEQ ID NO: 7 - DNA, 359 bp, Acanthamoeba castellanii
atggaaaagagggaggcacaggtggcacgagagacgggggagaccaagatcgaggtgcgcctgtcgctcgacggcacgggcgtgtccgatgtgaagaccggcatcggcttcctggaccacatgctgtccgccctcgccaagcacggtcgcttcgatctctatctccgatgcgcgggcgatctccacgtcgacgaccaccacacctccgaggactgcgcgatcgtgctgggccaagccttccgccaggccatcggtgagcgtaagggcatcaagcggtacgggtcggcctacgcgccgctggacgagagcctggcgcgcgccgtggtggacatctcgtctcccccgtctctcgcgaatct

SEQ ID NO: 8 - DNA, 297 bp, Acanthamoeba castellanii
acatgctcgccggtccctccgagtgcctcgtgctagccgacggcacggcccacccggacgtcgtggcggccgacctgctggcccaggccgagcacgacgtggcggccgtgcccatcttggtggcctacgacgaggagaccgtgcgccgggtcgaacaggaggtcgaggctcagctcgccaccctgcccaccgctgccatcgcccgcgccgccttctccaacggttttgctgtgattgtcgcgaacgaagaggaggccatcacggtgtgcgacaagctcgcgcccgagcacttggagg

SEQ ID NO: 9 - DNA, 21 bp, Artificial Sequence
  oligonucleotide A. castellanii CGS forward primer
atttggatggagtcgccgacc

SEQ ID NO: 10 - DNA, 19 bp, Artificial Sequence
  oligonucleotide A. castellanii CGS reverse primer
cttggtcaccgagtggacg

SEQ ID NO: 11 - DNA, 23 bp, Artificial Sequence
  oligonucleotide MS1 forward primer
aagcgcgaggggatgaggccgag

SEQ ID NO: 12 - DNA, 21 bp, Artificial Sequence
  oligonucleotide MS1 reverse primer
gtcgccacgggcaccatcggc

SEQ ID NO: 13 - DNA, 20 bp, Artificial Sequence
  oligonucleotide MS2 forward primer
tgccagacgttgcgggcatc

SEQ ID NO: 14 - DNA, 19 bp, Artificial Sequence
  oligonucleotide MS2 reverse primer
ccagggcgctgcacatgac

SEQ ID NO: 15 - DNA, 21 bp, Artificial Sequence
  oligonucleotide MS5 forward primer
gactcgtactcggcccgcagg

SEQ ID NO: 16 - DNA, 19 bp, Artificial Sequence
  oligonucleotide MS5 reverse primer
atcgacgcgctcgatgccg

SEQ ID NO: 17 - DNA, 174 bp, Acanthamoeba castellanii
atttggatggagtcgccgaccaacccgaccctcaagctcaccgacatccgcaaggtggcggaggtggccaagaagaggaacatcctgctggtcgtggacgacaccttcatgtcgccctacttccagaaccccctcgacctcggcgccgacatcgtcgtccactcggtgaccaag

SEQ ID NO: 18 - DNA, 188 bp, Acanthamoeba castellanii
gttgatgacatcatcagcaagctcgccgccatccgcgcgtcgttcgagggcaagctggtggtccagccctcggcgtcgcttcagcacgtgccctacgatgccaccgtggagaccaagctgccctcggccctgcgccaggtccttgccttcgccttccagaagctcaaggaggtcgacttcatcgctag

SEQ ID NO: 19 - DNA, 245 bp, Acanthamoeba castellanii
gtcgccacgggcaccatcggcttcccgcgtgtgggcaaggaccgagagcttaagaaggcgctcgagtcctactgggccggcaccaccgaccgtacccatttgctcggcgtggccgcccaggtcgagaaggccaacctcctcgcccaggtcgacgccggcatcgagcgcatcggcgtaggcagcttcagcctctacgaccaggtcctcgactgggccttccgcctcggcctcatcccctcgcgctt

SEQ ID NO: 20 - DNA, 308 bp, Acanthamoeba castellanii
atcgacgcgctcgatgccgatgtgatctcgatcgagaactcgcgtaacaacgacaagacgattaaggacctcaccgcctacggctacgagcgcgacatcggccccggcgtgtacgacatccacagcccggtggtgcccaccgtcgacgagattgtcgagaagctgcgcctcttcctcaagcacctccacccgtcgcgcgtcgtggtcaaccccgactgcggcctcaagacccgccagtggaaggaggtcgtgccgtcgctgcgcaacatggtcaccgccgccagcatcctgcgggccgagtacgagtc

SEQ ID NO: 21 - protein, 181 aa, Acanthamoeba castellanii
MEKREAQVARETGETKIEVRLSLDGTGVSDVKTGIGFLDHMLSALAKHGRFDLYLRCAGDLHVDDHHTSEDCAIVLGQAFRQAIGERKGIKRYGSAYAPLDESLARAVVDISSRPFAVIDLKLKREKIGELSCEMIPHVLHSFATSANLTLHVEVRHRVWCACGCVCVCVCVCVIVRVRVR

SEQ ID NO: 22 - protein, 247 aa, Aspergillus nidulans
MPLPARTASLSRNTNETKIQVSLSLDGGVLPPYEPSSHFPVPTDPLEAEAAKKGIVPNKDAAHATQFTPTQQITISTGIGFLDHMLHALAKHSGWSLAIRAKGDLYIDDHHTTEDTFLALGTAFTTALGARQSLARFGRGDAPLDEALSWAVIDLSSRPWAVINIGFKREKIGDLSTEMITHGLQSFAQAAGVTLHIGCTYGDNDHHRAESAFKALAVAIRAACARRVEGEVGAGDVSGTKGPALIE

SEQ ID NO: 23 - protein, 292 aa, Oryza sativa
MTTARFVSPSLSRVSPSPAGRVSGSSWLSRAGVALPARPHGLSLHLRPPAMASAAAAGNGSPSAPEDSTALSRIGEVKRVTKETNVHVKINLDGTGVADCSTGIPFLDHMLDQLASHGLFDVCVKAKGDTHIDDHHSNEDIALAIGTALLEALGDRKGINRFGHFTAPLDEAAVEVILDLSGRPHLSCGLSIPTERVGTYDTQLVEHFFQSLVNTSGMTLHIRQLAGKNSHHIIEATFKAFARALRQATEYDLRRRGTVPRLQYSKETNGMASTAPRFLLTLGISVGCDNRT

SEQ ID NO: 24 - protein, 220 aa, Asphodelus aestivus [sequence omitted]

SEQ ID NO: 25 - protein, 244 aa, Aspergillus fumigatus
MPLPARTATVSRVTNETKIQVSLSLDGGVLPPYEPSDHFPAPEDLKEAEAAKHGIVPPKNAAHATQFTPTQQITVSTGIGFLDHMLHALAKHSGWSLAIRAKGDLYIDDHHTTEDTFLALGTAFTKALGARQSLARFGRGDAPLDEALSWAVIDLSSRPWAVINLGFKREKIGDLSTEMITHGLHSFAQAADVTLHVGCTYGDNDHHRAESAFKALAVAIRTACTRRVAGEVGAGDVVSTKGVL

SEQ ID NO: 26 - protein, 272 aa, Arabidopsis thaliana
MELLSSSPAQLLRPNLSSRALLPPRTSIASSHPPPPRFLVMNSQSQHRPSISCASPPPGDNGFPAITTASPIESARIGEVKRETKETNVSVKINLDGHGVSDSSTGIPFLDHMLDQLASHGLFDVHVRATGDTHIDDHHTNEDVALAIGTALLKALGERKGINRFGDFTAPLDEALIHVSLDLSGRPYLGYNLEIPTQRVGTYDTQLVEHFFQSLVNTSGMTLHIRQLAGKNSHHIIEATFKAFARALRQATESDPRRGGTIPSSKGVLSRS